(19) United States
(12) Patent Application Publication
(10) Pub. No.: US 2026/0212949 A1
(43) Pub. Date: Jul. 23, 2026

(54) DIGITIZED COUNTERDIABATIC QUANTUM OPTIMIZATION METHOD FOR PROTEIN FOLDING

(71) Applicant: Kipu Quantum GmbH, Karlsruhe (DE)

(72) Inventors: Enrique Solano, Berlin (DE); Narendra Hegade, Karnataka (IN); Iraitz Montalban, Santander (ES)

(73) Assignee: Kipu Quantum GmbH, Karlsruhe (DE)

(21) Appl. No.: 19/137,109

(22) PCT Filed: Dec. 14, 2023
(86) PCT No.: PCT/EP2023/085969

§ 371 Date: Jun. 9, 2025

(30) Foreign Application Priority Data
Dec. 14, 2022 (EP) 22213554.3
Jun. 20, 2023 (EP) 23020302.8

(51) Int. Cl. G16B 15/20; G06N 10/20; G06N 10/60

(52) U.S. Cl. CPC G16B 15/20; G06N 10/20; G06N 10/60

(57) ABSTRACT

The invention relates to a computer-implemented method for solving an optimization problem for the folding of a protein with a given amino acid sequence wherein a Hamiltonian function encoding the problem is to be minimized. The method comprises the following steps:—choosing a lattice model,—calculating the amino acids of the given protein sequence in the lattice model such that the interaction energy of the amino acids is minimized, thereby creating a problem Hamiltonian whose ground state shows the configuration of the concerned protein in the given lattice, and—performing an optimization routine to obtain the final Hamiltonian in the ground state reflecting the solution to the optimization problem.

### CROSS-REFERENCE TO RELATED APPLICATIONS

This application claims priority to European Patent Application No. 22213554.3, filed Dec. 14, 2022, and to European Patent Application No. 23020302.8, filed Jun. 20, 2023, the disclosures of which are incorporated by reference herein in their entirety.

### FIELD OF THE INVENTION

The invention relates to a method for optimizing protein folding. The invention provides a protein folding method, and belongs to the technical field of quantum computing. Certain aspects of the invention are defined in the claims.

### DETAILED DESCRIPTION OF EXEMPLARY EMBODIMENTS OF THE INVENTION

Proteins are (macro)molecules consisting of a chain of amino acid residues and perform many vital functions in organisms like DNA replication, catalyzing metabolic reactions, etc. Knowledge of how proteins fold is crucial in understanding many enzymes, and also, the mechanics of folding may unravel remedies for diseases like Alzheimer's, Huntington's, and Parkinson's that are induced due to misfolding of proteins. With the exponentially increasing conformational energy landscape, the protein folding problem is a highly complex problem to solve using classical computational techniques, which inspires the use of quantum computation. Protein folding problems are reduced to an optimization problem by suitable encoding and investigated using quantum computers. Protein folding problems entail finding the optimal protein configuration.

Here, digitized-counterdiabatic quantum computing is used to tackle optimization problems using counterdiabatic protocols in an adiabatic quantum optimization.

The invention uses a hybrid quantum-classical digitized-counterdiabatic quantum algorithm to tackle protein folding problems. Finding the lowest energy configuration of an amino acid sequence is an NP-hard optimization problem that plays a prominent role, for example, in drug design applications, biology and chemistry. A main feature of the invention is that the method is hardware-implementable, which is in contrast to the general nature of algorithms implementing problem-inspired quantum circuits.

The method/algorithm is implemented for proteins on simulators, quantum computer emulators, and currently available quantum hardware platforms based on trapped-ions and superconducting circuits.

The object of the present invention is to provide a way to solve a protein folding optimization problem. This object is achieved by the invention.

In one aspect, the invention refers to a method, in particular a computer-implemented method, for solving an optimization problem for finding a solution of a protein folding problem.

In certain embodiments, the invention refers to a computer-implemented method for solving an optimization problem, in particular an adiabatic quantum optimization problem, for the folding of a protein with a given amino acid sequence, wherein a Hamiltonian function, in particular an adiabatic Hamiltonian function (also referred to here as Hamiltonian) encoding the problem is to be minimized.

A protein in the context of the invention is a molecule comprising a plurality of amino acids. The amino acids may be naturally occurring amino acids or artificial amino acids.

In certain embodiments, the method may comprise the following steps:
providing a lattice model (also known as lattice point model), in particular a suitable 2D or 3D lattice model,
placing the amino acids of the protein sequence in the lattice model, such that an interaction energy of the amino acids is minimized, thereby creating a problem Hamiltonian (whose ground state shows the configuration of the protein in the lattice), and
performing an optimization, in particular a reiterative optimization on the problem Hamiltonian to obtain a final Hamiltonian in the ground state reflecting the solution to the optimization problem.

The problem Hamiltonian and an initial Hamiltonian are both part of an adiabatic Hamiltonian (see Eq. 6).

In certain embodiments of the method of the invention, an initial Hamiltonian is chosen. In particular, a initial Hamiltonian is chosen whose ground state can be easily prepared, as known in the art. The system is then subject to a time-dependent adiabatic process where the initial Hamiltonian gradually transforms into the final Hamiltonian. The final Hamiltonian corresponds to the problem Hamiltonian encoding the solution to the protein folding problem.

A Hamiltonian is said to be k-local if it involves at most k qubits, where k is an integer number.

In certain embodiments of the method, a smooth time dependent scheduling function is chosen and the adiabatic Hamiltonian is defined using the initial Hamiltonian, the final Hamiltonian and/or a scheduling function.

In certain embodiments, the method comprises calculating a set of (approximate) couterdiabatic (CD) terms (k-local terms with k being an integer) for the adiabatic Hamiltonian, for example, by using a nested commutator method. Subsequently, from the obtained set of CD terms, a set of operators from the obtained set of CD terms is selected. The CD term selection can depend on the hardware constraints. For example, if the hardware has only local connectivity one can rely on local CD terms, if the hardware offers non-local connectivity, one can consider non-local higher order CD term. The selected CD terms can later be implemented on a given quantum processor.

Based on the hardware constraints, the CD terms are selected from the set and a variational parameter is associated for each CD term.

If the hardware has only local connectivity, as in superconducting QCs, one can use on local CD terms. If the hardware offers non-local connectivity (as, for example, in a trapped-ion system) one can use non-local higher order CD terms.

In certain embodiments of the method, only 2-local terms are selected as (approximate) couterdiabatic CD terms and higher order terms are not chosen. In other embodiments, however, not only 2-local terms but also higher order terms are selected, such as 3-local terms.

In certain embodiments, a circuit ansatz is constructed using the selected CD terms. The circuit ansatz with the CD terms may be mapped to a given hardware based on the hardware connectivity/qubit connectivity. SWAP methods and circuit optimization techniques are optionally used to optimize the circuit depth.

SWAP methods use SWAP gates (that are used to optimize quantum circuits, in terms of circuit depth. A SWAP gate is a quantum operation that exchanges the states of two qubits. It is used to perform an operation on two qubits that do not have a direct connection in the quantum computer.

Circuit optimization techniques are strategies used to reduce the complexity of a quantum circuit, making it more efficient for running on a quantum computer. These techniques are useful in running algorithms of Noisy Intermediate-Scale Quantum (NISQ) devices, which have limited qubit counts, connectivity, and coherence times. Examples of such techniques include gate reduction, gate decomposition which involves decomposing multi-qubit gates into hardware compatible gates, SWAP methods, parallelization involving executing as many quantum gates in parallel and error mitigation or correction techniques.

Subsequently, a variational minimization may be performed in certain embodiments to minimize the expectation of the problem Hamiltonian using a gradient-based or a gradient-free classical optimizers.

A variational minimization involves using variational principle used in algorithms such as the Variational Quantum Eigensolver (VQE). The VQE is a hybrid quantum-classical algorithm that aims to find the ground state energy of a quantum system, which is equivalent to finding the minimum eigenvalue (energy) of the system's Hamiltonian.
The variational minimization procedure is repeated for multiple random initial parameters, and the best solution is post-selected.
From the obtained solution/bit string from the quantum computer, the Euclidean coordinates are obtained using known techniques, and the final form of the folded protein is acquired.

In certain embodiments, the invention refers to a method, wherein the selected (approximate) couterdiabatic (CD) terms are used to construct a variational quantum circuit in the form of a parametrized quantum circuit (PQC) comprising a set of quantum gates. In these embodiments, the circuit ansatz consists only of couterdiabatic (CD) terms, i.e. no terms from the adiabatic Hamiltonian are included, which are only used for calculating the CD terms. This greatly reduces the circuit depth compared to a quantum approximate optimization algorithm (QAOA) ansatz as well as other previously proposed CD protocols and lead to an advantage over these known methods.

In certain embodiments, the parametrized quantum circuit (PQC) comprises of at least one (i.e. one or more) variational parameter that is/are optimized using an optimizer such as a gradient-based optimizer or a gradient free optimizer in an iterative fashion.

In certain embodiments, a hybrid quantum-classical digitized-counterdiabatic quantum algorithm is applied, in particular comprising a parameterized quantum circuit (PQC) including at least one CD term and, optionally a classical optimization routine for optimizing variational parameters of the parameterized quantum circuit.

In certain embodiments, the invention refers to a method, comprising generating quantum states based on optimal parameters provided by at least one classical optimization routine, wherein the classical optimization routine can be a gradient-based optimizer or a gradient-free optimizers, configured to minimize a cost function C that is the expectation value of the problem Hamiltonian.

In some embodiments, at each iteration of the variational optimization, the energy expectation value corresponding to the problem Hamiltonian with respect to a trial state is measured and the at least one variational parameter is updated in order to minimize the expectation value until a convergence is reached or at a pre-defined number of maximum iterations has been reached.

In certain embodiments, the energy expectation value of the problem Hamiltonian is minimized, with the energy expectation defined as

$$〈 {H}_{p} 〉 = 〈 {ψ}_{trail} \vert {H}_{p} \vert {ψ}_{trail} 〉 ,$$

wherein Hₚ is the problem Hamiltonian and |ψₜᵣᵢₐₗ [symbol] as a trial wavefunction prepared by the CD ansatz as described herein. A trial wavefunction is an approximate wavefunction that is used as an initial estimation or ansatz for the true wavefunction of a system.

The iterative optimization is performed until the convergence or a pre-defined number of maximum iterations is reached.

In some embodiments, the final solution after the optimization is determined by measuring the qubits of the quantum processor in the computation Z basis. Here, the term “Z basis” refers to the standard computational basis for qubits with the two states |0> and |1>. From the obtained solution, one can determine the protein configuration on the point lattice sites, e.g. the 2D or 3D lattice sites.

In certain embodiments of the computer-implemented method of the invention for solving an optimization problem for the folding of a protein with a given amino acid sequence, the protein folding problem is encoded into a computable higher order unconstrained binary optimization problem (HUBO form), and a lattice point model is adopted to simulate the composition of amino acid polypeptide chain molecules in a three-dimensional space.

A Higher Order Unconstrained Binary Optimization (HUBO) problem is an extension of the Quadratic Unconstrained Binary Optimization (QUBO) problem. It is a type of optimization problem where the objective function is a polynomial of binary variables (where each variable can take a value of 0 or 1) and can include terms that are of higher order than two. This means that the function to be minimized or maximized includes terms that are the product of three or more binary variables.

In certain embodiments, the invention refers to a computer-implemented method, comprising one or several of the following steps:
Choosing a lattice structure on which the protein folding is modeled, for example, a 3D tetrahedral lattice model.
After selecting the lattice structure, choose an encoding type, which can be, for example, position encoding (the lattice coordinates are encoded as qubits), or turn encoding (the turn direction is encoded as qubits).
Based on the interaction between the amino acids and the imposed constraints, the problem Hamiltonian is constructed, whose ground state is the solution to the protein folding problem.
In order to find the ground state of the problem Hamiltonian, a hybrid classical-quantum optimization method is performed.

Moreover, the method may comprise at least one of the following steps in certain embodiments (in particular with respect to placing amino acids of the protein sequence in a lattice model):
coding binary amino acid sequences, defining the step length and coordinates of any amino acid in the coded binary amino acid sequences in a predetermined direction and the distance between any two amino acids,
constructing a corresponding Ising Hamiltonian by adding constraint conditions, and
evolving the Ising Hamiltonian by utilizing quantum annealing to obtain the final evolution result, wherein the energy optimal solution obtained through the evolution is the most stable conformation of the protein.

In certain embodiments, the invention refers to a computer-implemented method for modeling a three-dimensional protein structure, the method comprising:
receiving a primary amino acid sequence of a three-dimensional protein; translating the primary amino acid sequence to a first vector, wherein the first vector comprises a unique numerical descriptor value corresponding to each amino acid residue in the primary amino acid sequence;
determining a per-residue conformation index for each amino acid residue in the primary amino acid sequence;
determining a vector set for each amino acid residue in the primary amino acid sequence, wherein the vector set comprises a plurality per-residue interaction factors corresponding to a plurality of conformation indexes for that amino acid residue; and
using the per-residue interaction vector set to generate a multi-dimensional matrix for the three-dimensional protein structure.

In certain embodiments, the invention refers to a computer-implemented method, in particular to a method of operation in a hybrid quantum-classical computational system, the method comprising:
receiving an optimization problem; and
for a number of iterations i to a number n; where n is a positive integer:
causing a solver in the form of a quantum annealer that has a schedule function that is parameterized for at least one tunable parameter to be executed by at least one quantum processor to generate a plurality of samples as potential solutions to the optimization problem; causing, by at least one controller, a performing of at least one post-processing operation on the plurality of samples by at least one postprocessing quantum processor-based device to generate a set of postprocessing results;
determining whether to modify the optimization problem based at least in part on the set of post-processing results;
upon determining to modify the optimization problem based at least in part on the set of post-processing results, the i^{th} iteration further comprising:
causing the optimization problem to be modified; and
initiating an (i+1)^{th} iteration.

In certain embodiments, the invention refers to a use of the method described herein in quantum chemistry, in particular for solving a protein optimization problem as described herein, with the protein having a given amino acid sequence, wherein a Hamiltonian encoding the problem is minimized.

In certain embodiments, the invention refers to a computer program having a program code for performing the method described herein, when the computer program is executed on a computer, a processor, a quantum processor and/or a programmable hardware component.

In certain embodiments, the invention refers to a computation device comprising: an interface for communicating with a quantum-processing unit; and one or more processors configured to perform the method described herein using the quantum processor.

In another aspect, the invention refers to a computer program having a program code for performing the method as described herein, when the computer program is executed on a computer, a processor, a quantum-processing unit and/or a programmable hardware component.

In another aspect, the invention refers to a computation device comprising: an interface for communicating with a quantum-processing unit; and one or more processors configured to perform the method as described herein using the quantum-processing unit.

In another aspect, the invention refers to an apparatus comprising:
a quantum device; and one or more computing devices communicatively coupled with the quantum device; the one or more computing devices being configured to at least cause the apparatus to: provide a quadratic unconstrained binary optimization problem defined by an equation with a cost function for optimization; and introduce a first set of data into the problem, the first set of data comprising historical financial data for a first period of time, the historical financial data at least comprising prices of considered assets; the quantum device being configured to at least cause the apparatus to solve the quadratic unconstrained binary optimization problem for the first period of time, thereby obtaining optimal trajectories for the first period of time; and the one or more computing devices being configured to at least further cause the apparatus to: provide a quantum or classical machine learning algorithm that provides a recommended composition based on a set of inputs; train the machine learning algorithm by both inputting the optimal trajectories obtained by the quantum device for the first period of time and minimizing a predetermined error function for each time unit of the first period of time for which there is historical data in the first set of data; introduce a second set of data into the machine learning algorithm, the second set of data comprising data for a second period of time that is posterior to the first period of time; and provide a recommended composition for the second period of time by running the trained machine learning algorithm with the second set of data introduced therein.

In another aspect, the invention relates to a data processing apparatus/device/system comprising means for carrying out the method of the invention as described herein.

In another aspect, the invention refers to a system for performing a method as described herein, comprising
a quantum processor with tunable coupling,
a memory to save the results of the measurements of expectation values of a final Hamiltonian, and
a classical processor for performing a classical optimization.

The quantum processor is tunable because it allows for fitting parameters of the parametrized quantum circuit (PQC). The couterdiabatic terms are used to construct a variational quantum circuit in the form of a parametrized quantum circuit (PQC) comprising a set of quantum gates.

In another aspect, the invention relates to a computer program (product) comprising instructions which, when the program is executed by a computer, cause the computer to carry out the method of the invention as described herein. The invention also refers to a computer program having a program code for performing the method of the invention, when the computer program is executed on a computer, a processor, a quantum processor and/or a programmable hardware component.

In another aspect, the invention relates to a computation device comprising: an interface for communicating with a quantum-processing unit; and one or more processors configured to perform the method as described using the quantum processor.

In another aspect, the invention relates to a computer-readable data carrier or storage medium having stored thereon said computer program (product). In another aspect, the invention relates to a computer-readable [storage] medium comprising instructions which, when executed by a computer, cause the computer to carry out [the steps of] the method as described.

### Definitions

An optimization problem is a mathematical problem where the task is to find the parameters that minimize or maximize a given multivariable function, normally called the cost function.

Optimization algorithms may include simulated annealing, parallel tempering, Markov Chain Monte Carlo techniques, branch and bound algorithms, and greedy algorithms, which may be performed by a classical computer. Optimization algorithms may also include algorithms performed by a quantum computer, such as quantum annealing, quantum approximate optimization algorithm (QAOA) or other noisy intermediate-scale quantum (NISQ) algorithms, quantum implemented fault-tolerant optimization methods, or other quantum optimization algorithms.

A general parametrization of the schedule function may be performed using several ways, including several time-dependent tunable parameters in the adiabatic quantum dynamics.

The final Hamiltonian (also referred to here as problem Hamiltonian) is the Hamiltonian that is addressing a time t=T in adiabatic quantum computing, quantum annealing, and by quantum annealers. The ground state of this Hamiltonian codifies the solution of an optimization problem.

A schedule function is a function that interpolates the initial and final Hamiltonian in adiabatic quantum computing and quantum annealing, and needs to be experimentally realizable by a quantum annealer.

Parameters are “tunable” if they are experimentally manipulable during the course of the method. Tunable coupling refers to the tunable parameter that is related to the physical interaction between two or more informational units (quantum bits).

An expectation value is the mean value obtained after an experimental measure of a physical quantity several times in a quantum experiment.

A quantum processor is a programable quantum devices composed of several informational units (qubits) that can be tuned in order to perform quantum algorithms.

In certain embodiments, the invention is used in chemistry to solve optimization problems. Such chemical optimization problems may be optimization problems for finding the ground state (the lowest energy state) of a chemical molecule such as a (poly)peptide or protein. The method of the invention is general and can be applied to any molecular ground state calculations of (poly)peptides and proteins, provided that they form a stable low energy conformation. The size of the molecule may be in the range of few tens of atoms to a few hundred atoms. In some embodiments, the molecule has 20 to 500 amino acids, in particular 30 to 150 amino acids. In some embodiments, the peptide chain of the molecule has 5 to 29, in particular 7 to 9 amino acids.

Quantum computers (quantum processors) may include quantum annealing processors, digitized quantum processors, gate-based processors, or adiabatic quantum computation. On successive iterations the incremented optimization algorithm may provide samples, including quantum annealing, gate model-based processors, etc.

A solver is a mathematical-based set of instructions executed via hardware circuitry that is designed to solve mathematical problems. Some solvers are general purpose solvers, designed to solve a wide type or class of problems. Other solvers are designed to solve specific types or classes of problems. A non-limiting exemplary set of types or classes of problems includes: linear and non-linear equations, systems of linear equations, non-linear systems, systems of polynomial equations, linear and non-linear optimization problems, systems of ordinary differential equations, satisfiability problems, logic problems, constraint satisfaction problems, shortest path or traveling salesperson problems, minimum spanning tree problems, and search problems.

There are numerous solvers available, most of which are designed to execute on classical computing hardware, that is computing hardware that employs digital processors and/or digital processor-readable nontransitory storage media (e.g., volatile memory, non-volatile memory, disk based media). More recently, solvers designed to execute on non-classical computing hardware are becoming available, for example solvers designed to execute on analog computers, for instance an analog computer including a quantum processor.

A method of operation in a computational system, may be summarized as including: receiving a problem and performing a number of iterations i to a number n, where n is a positive integer. Each iteration includes causing a solver to be executed by at least one processor to generate a plurality of samples as potential solutions to the problem; causing, by at least one controller, a performing of at least one post-processing operation on the plurality of samples by at least one post-processing non-quantum processor-based device to generate a set of post-processing results; and determining whether to modify the problem based at least in part on the set of post-processing results. Upon determining to modify the problem based at least in part on the set of post-processing results, the ith iteration further includes causing the problem to be modified and initiating an (i+1)th iteration.

Causing the solver to be executed by at least one processor to generate a plurality of samples as potential solutions to the problem may include causing the problem to be optimized by at least one heuristic optimizer executed by at least one processor to generate a plurality of samples as potential solutions to the problem.

Determining whether to modify the problem based at least in part on the set of post-processing results may include comparing a result to a determined satisfaction condition and/or comparing the number of iterations performed to a determined limit.

In some implementations of the above-described method of operation in a computational system, the at least one processor is a quantum processor.

### FIGURES

FIG. 1: A Schematic diagram of different types of ansatz with p=1 layer, implemented in embodiments of this invention. These ansatz are sorted from left to right from hardware-efficient ansatz that has no information from the problem to QAOA which is a problem-inspired ansatz. The CD-inspired ansatz implemented in this work is shown in the middle. Horizontal lines show qubit registers. In all the cases, the initial state is |+ [symbol] ^{⊗}^{^{N}}. In hardware-efficient ansatz: (Eq. (2)), there are parameterized Y rotations followed by nearest-neighbor entangling gates then again parameterized Y rotations, in QAOA ansatz (Eq. (4)): Hamiltonian term U_{c}(γ) and mixer term U_{b}(β) and in CD-inspired ansatz (Eq. (8)): Parameterized Y rotations followed by YZ rotations. In all the cases, cost function C (Eq. (1)) is computed then the parameters are updated using gradient-based classical optimizers until C is minimized. Going from left to right, the hardware-implementation becomes more difficult.

FIG. 2: Schematic diagram showing the tetrahedral lattice structure. (a) shows the lattice P, (b) shows the inverted lattice Q and (c) shows the mesh created by the tetrahedral lattice with red colored lines showing the turns taken by an arbitrary amino acid chain.

FIG. 3: Success Probability as a function of 20 randomly initialized instances for 6, 7 and 8 amino acid proteins with N=6, N=9 and N=13 respectively using the CD-inspired ansatz with p=1 layer and maximum 500 iteration steps. Results are obtained with p=1 layered circuit and gradient-based optimizers Adam and Adagrad were used for classical optimizations.

FIG. 4: Energy as a function of iterations for 13 qubit protein folding problem. Results show simulator data for 100 iterations using Adam optimizer comparing QAOA and CD-inspired ansatz. This is the performance of the best instance out of the 20 randomly initialized parameter instances.

FIG. 5: Energy as a function of iterations for N=17 qubits system comparing CD-inspired ansatz and hardware-efficient ansatz. Numerical simulations were performed for 100 iterations with Adam optimizer. Inset plot shows the minimum energy achieved during the optimization.

FIG. 6: Energy as a function of iterations for N=9 qubits system with noise model mimicking the noise of ibmq guadalupe. Numerical simulations were performed for 100 iterations with Adagrad optimizer for 5 instances. Blue dots show the average of these instances and red shaded region shows the standard deviation. The inset shows the last 10 iteration steps. Green line shows the exact ground state energy.

FIG. 7: Output probability distribution of N=13 AVDINNNA protein and N=17 CYIQNCPLG protein on a trapped-ion system: Quantinuum system H1 with 1000 shots. (a) Shows the N=13 case and (b) Shows the N=17 case. (a1) and (b1) show the graph corresponding to two-body interactions implemented in the CD-inspired ansatz. Blue edges show the present two-body connections while the purple edges show the connections that are absent. (a2) shows the optimal protein configuration with a dotted green line depicting the connection of the nearest-neighbor interaction. (b2) show the optimal configuration of protein obtained from exactly solving the problem whereas (b3) shows the protein configuration obtained in the experiment. In (b3) amino acids ‘P’ and ‘G’ are overlapping.

FIG. 8: Output probability distribution of N=9 qubits by implementing the optimal circuit on (a) IBM's ibmq guadalupe where the experiment was performed with 8192 shots and (b) Google's quantum virtual machine rainbow where the experiment was performed with 10000 shots. Dark-colored bars show the ground-state probability of the physical qubits whereas light-colored bars show the rest of the distribution (a1) and (b1) show the hardware topology and selected qubits are shown using red color. (a2) and (b2) both show the optimal protein configurations with the nearest neighbor connection between ‘A’ and ‘F’ shown by a dotted green line.

FIG. 9: Number of free parameters for hardware-efficient ansatz, CD-inspired ansatz for this work, and CD-inspired ansatz where all-to-all two-body interactions are present.

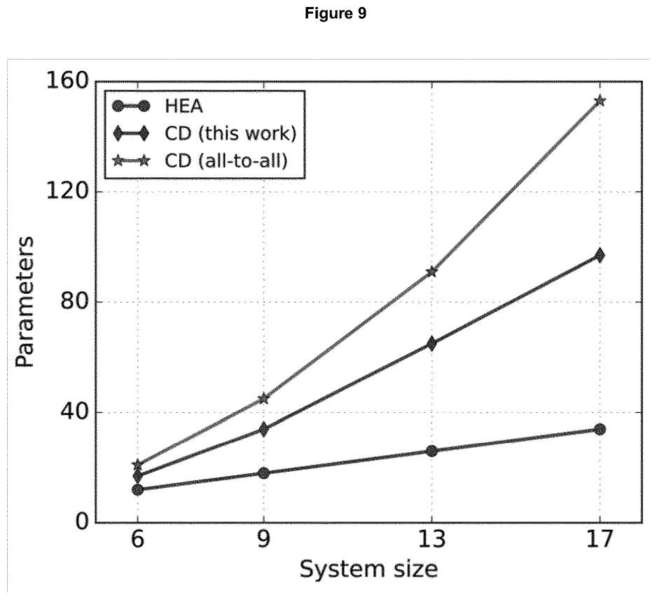

FIG. 10: Native gate decomposition of YZ gate in real hardware for qubits i and j. (a) shows the decomposition for Quantinuum tapped-ions hardware (native gate ZZ), (b) shows the decomposition for IBM superconducting hardware (native gate CR) and (c) shows the decomposition for Google QVM (native gate CZ).

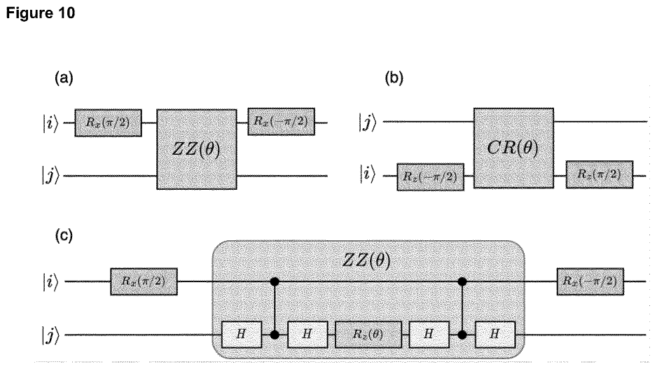

FIG. 11: Error map of IBM's 16 qubit ibmq guadalupe device when the experiment described in the Examples was performed.

### EXAMPLES

I. Variational quantum algorithms (VQAs) have gained attention as a useful application for the near-term era. VQAs are hybrid classical-quantum algorithms implemented to optimize a cost function that contains information about the solution. The quantum part of a VQA consists of a parameterized quantum circuit (PQC) (also known as circuit ansatz) to produce quantum states and the classical part consists of an optimization routine that gives optimal parameters to solve the problem. The choice of PQC affects the performance of the VQA to a great extent. These PQCs are broadly divided into two categories: Problem-inspired and hardware-efficient. Problem-inspired ansatz utilizes the properties of the problem Hamiltonian to efficiently reach the expected state while hardware-efficient ansatz takes the information of the device connections to reduce the noise due to deep circuits and unimplementable connections. Some examples of problem-inspired ansatz are the Quantum Approximate Optimization Ansatz (QAOA), the unitary coupled cluster ansatz or Hamiltonian variational ansatz; while on the other hand some are noteworthy hardware-efficient ansatzes.

Apart from the successes, implementing VQAs comes with certain challenges such as noise, a limited number of shots, etc. It has also been shown that VQAs suffer from barren plateaus, where the gradients vanish with increasing system size. Generally, VQAs with hardware-efficient ansatz suffer from this challenge due to their high expressibility. Hence the use of the problem-inspired ansatz is motivated. In problem-inspired ansatz, the idea is to take information from the problem, which constrains the energy landscape that results in lower expressibility and higher trainability. In any case, the ansatz should be trainable enough so that using classical optimization, one can reach the solution effectively in fewer iterations. That being said, problem-inspired ansatzes usually are very deep, so experimental implementation becomes unfeasible. Thus a “good” circuit ansatz has to be expressible so that it contains the solution but not too expressible that it leads to barren plateaus and has to be trainable enough to reach the solution.

Recently, works have reported the use of “digitized-counterdiabatic quantum computation (DCQC)” to improve the state-of-the-art quantum algorithms. As the name suggests, these methods utilize digitized-counterdiabatic (CD) protocols to improve the quantum algorithms like digitized adiabatic evolution, quantum approximate optimization algorithm (QAOA), etc. and have already shown drastic improvements in industrial applications like portfolio optimization, and factorization. With the advantages, these methods come with challenges like finding suitable initial parameters and finding optimal CD driving terms. There have been several attempts to solve these challenges, for example, a meta-learning technique was proposed recently to find suitable initial parameters and the choice of optimal CD terms by machine learning methods like reinforcement learning and Monte-Carlo tree search was also proposed. Optimizing CD terms using variational quantum circuits has also been studied. CD driving falls under the umbrella of the shortcuts to adiabaticity methods that were developed to accelerate the quantum adiabatic processes. Among many methods, like fast forward and invariant-based engineering, CD driving has been of prominent interest over the years for studying many-body quantum systems and has shown significant results.

The inventors developed this paradigm by implementing a hybrid quantum-classical digitized-counterdiabatic quantum algorithm to tackle a protein folding problem. This algorithm consists of a PQC inspired by CD driving and a classical optimization routine for parameter optimization.

Generally, protein folding is done by using lattice point models, in which a suitable 2D or 3D lattice is chosen and the amino acids are allowed to be placed in it such that the interaction energy is minimized. By proper encoding schemes, this problem can be converted into a problem Hamiltonian whose ground state shows the configuration of the concerned protein in the given lattice.

This problem was investigated by using a PQC which is called herein “CD-inspired ansatz”. While being problem-inspired, this ansatz is also hardware implementable and has a parameterization that scales with the [symbol] (N²). These features along with other salient features make this ansatz suitable for this problem. In the next sections, the construction of the ansatz is explained, and to analyze the performance, ansatz is applied to various proteins up to a size of N=17 qubits using ideal simulators and noisy simulators. The inventors also implement this ansatz to several real devices with different device connectivity and native gates, for instance, the 1D chain connection of IBM superconducting chip, 2D grid connection of Google's superconducting quantum virtual machine, and all-to-all connection of Quantinuum's trapped ions.

In the next section, VQA and counterdiabatic driving are recapitulated. In section III, the CD-inspired ansatz is explain in detail. In section IV, the preliminaries of the protein folding problem are provided. In section V, this ansatz is applied to various problems with ideal simulator and noisy simulator. In section VI, experiments performed on several real hardware are described and finally section VII is devoted to discussions.

### II. Variational Quantum Algorithms and Counterdiabaticity

In VQA, a circuit ansatz and a classical optimizer combine to solve an optimization problem. The task of the circuit ansatz is to generate quantum states based on the optimal parameters provided by the classical optimization routines. The classical optimizations can be gradient-based optimizers (like Adam or Adagrad) or gradient-free optimizers (like Cobyla or Powell). The goal is to minimize a cost function C that can take various forms depending upon the problem but in general, it is the expectation value of the problem Hamiltonian H given by,

$$C ( θ ) = 〈 ψ ( θ ) \vert H \vert ψ ( θ ) 〉 , \qquad ( 1 )$$

where θ={Σθᵢ} shows the parameters associated with the circuit ansatz. As mentioned earlier, both problem-inspired ansatz and hardware-efficient ansatz have their advantages and disadvantages. Despite the promising performance of the problem-inspired ansatz, the current hardware experience several bottlenecks like limited qubit connectivity, imperfect implementation of gates, limited coherence times, etc. which makes the implementation impractical so hardware-efficient ansatz is implemented. Generally, the hardware-efficient ansatz is of the form,

$$U ( θ ) = {\sum }_{k = 1}^{p} {U}_{k} ( {θ}_{k} ) {W}_{k} \qquad ( 2 )$$

where θ={Σₖ θₖ} are the optimizable parameters. Uₖ=exp[−iθₖVₖ] where Vₖ is a hermitian operator. Wₖ are non-parameterized gates usually consisting of 2-qubit connecting gates like CNOTs and Uₖ are parameterized single qubit rotations. p is the number of layers.

On the other hand, the problem-inspired ansatz use evolutions of the form,

$$U ( θ ) = {e}^{- \hat{igt}} \qquad ( 3 )$$

where ĝ is a Hermitian operator and t is a parameter. These ĝ are derived from the system of interest, for instance, in QAOA, ĝ=H where H corresponds to the problem Hamiltonian, thus Eq. (3) resembles the trotterized-time evolution. In QAOA, the quantum circuit consists of two unitaries: Hamiltonian term U_{c}(γ) and mixing term U_{b}(β) applied p-times to the initial state |ψ₀ [symbol] =|+ [symbol] ^{⊗N} where (γ, β) are the parameters to optimize by classical optimizer. So, the evolution looks like,

$$\vert {ψ}_{f} 〉 = {U}_{b} ( {β}_{p} ) {U}_{c} ( {γ}_{p} ) {U}_{b} ( {β}_{p - 1} ) {U}_{c} ( {γ}_{p - 1} ) … {U}_{b} ( {β}_{1} ) {U}_{c} ( {γ}_{1} ) \vert {ψ}_{0} 〉 \qquad ( 4 )$$

where |ψ_{f} [symbol] shows the output state,

$${U}_{b} ( β ) = {e}^{- i β {\sum }_{i} {σ}_{i}^{x}}$$

and U_{c}(γ)=e^{-iγH}. The aim is to minimize the cost function given by Eq. (1). QAOA directly relates to the quantum adiabatic evolution hence it has believed to be a successful algorithm at large p layers due to the adiabatic theorem. That being said, implementing circuits with large p results in high circuit depths, not feasible for current near-term devices. Many adaptations to QAOA have been reported. Among them, CD driving has been of interest recently from which the newly proposed “digitized-counterdiabatic QAOA (DC-QAOA)” reports that the addition of CD terms to the usual QAOA ansatz gives significant improvements in the performance of QAOA. Finding CD terms is a critical task and is done by obtaining approximate CD terms by the adiabatic gauge potentials using the nested commutator (NC) method. In the NC method, the approximate CD terms are given by

$${A}_{λ}^{( l )}$$

and can be calculated as,

$${A}_{λ}^{( l )} = i {\sum }_{k = 1}^{l} {α}_{k} ( t ) [ {H}_{a} , [ {{H}_{a} \underbrace{,} …}_{2 k - 1} [ {H}_{a} , {\partial }_{λ} {H}_{a} ] ] ] \qquad ( 5 )$$

where l shows the order of expansion and Hₐ(t) is the standard adiabatic evolution given by,

$${H}_{a} ( t ) = ( 1 - λ ( t ) ) {H}_{m i x e r} + λ ( t ) H \qquad ( 6 )$$

where Hₘᵢₓₑᵣ is a Hamiltonian whose ground state is easy to prepare (also known as the initial Hamiltonian) and λ(t) is a scheduling function with boundary conditions λ(0)=0 and λ(T)=1, where T is the total evolution time. The CD term is then digitized and the coefficient α is considered as an additional free parameter along with (β, γ) to increase the ansatz expressibility. Summing up, DC-QAOA uses three unitary terms (instead of the conventional two) iteratively p-times: Hamiltonian term, mixer term, and CD term to minimize the cost function more effectively. The disadvantages that come with DC-QAOA ansatz are the increased circuit depth per layer and unfeasible experimental implementation for many-local Hamiltonians. To circumvent these issues, a CD-inspired circuit ansatz is described that is also hardware efficient and hence partakes the advantages of both problem-inspired and hardware efficient ansatz. In the next section, it will explained how to create such an ansatz and then benchmark its performance by applying it to the protein folding problem.

### III. CD-Inspired Ansatz

In this section, it will be explained how to construct a CD-inspired ansatz and also give reasons on why this kind of ansatz works for hybrid quantum-classical optimization. To do so, one starts by considering quantum adiabatic evolution with counterdiabatic driving Hamiltonian H_{cd} given by

$${H}_{cd} = {H}_{a} ( t ) + \dot{λ} ( t ) {A}_{λ} \qquad ( 7 )$$

where Hₐ(t) is given by Eq. (6) and A_{λ} is given by Eq. (5). When working with the adiabatic regime, the condition on the scheduling function λ(t) is that it is slow enough such that the adiabatic theorem is satisfied and one can reach the ground state of the target Hamiltonian. However, if the CD term as shown in Eq. (7), is added this condition is lifted as the non-adiabatic transitions are suppressed by the additional CD term and at |λ|→0, ones retrieve the adiabatic condition. Now considering a scenario where for a certain λ(t), one obtains |λ(t)|>>1. For this scenario, the evolution will almost be non-adiabatic and most of the contribution to the evolution will be from AA term. In theory, as the CD term suppresses the non-adiabatic transitions, the evolution should also be successful, but it will require the calculate of the exact CD term, that suppresses all the transitions. In DC-QAOA, the Eq. (7) is trotterized, to get faster evolution and instead of using actual scheduling functions, classical optimization routines can be used to optimize parameterized trotter evolution to reach to the ground state. Under the assumption that there exist a scenario as mentioned above, one can get rid of the contribution from Hₐ(t) and in the circuit ansatz, only the CD terms can be implemented. Instead of implementing all the evolution, just the contributions from the CD term are implemented as a parameterized circuit and allow the classical optimization to take care of the evolution to lead to the ground state. Thus, the CD-inspired ansatz will have the form,

$${U}_{cd} ( θ ) = {e}^{- i θ A} \qquad ( 8 )$$

where A∈A_{λ} and A_{λ} is a set of all the terms computed from Eq. (5). This is advantageous for VQAs in the sense that this condition gets rids of most of the terms from the ansatz which makes it implementable in the near-term devices and as these algorithms aim to find approximate solutions this ansatz should lead to good solutions if a suitable optimization strategy is used.

The quantum optimal control (QOC) theory is a framework to provide methodological manipulation of dynamical systems. In QOC,

$${H}_{QOC} = {\sum }_{m} {c}_{m} ( t ) {H}_{m} \qquad ( 9 )$$

where Hₘ are control Hamiltonians and cₘ(t) are control fields. Control Hamiltonians are usually non-commuting [Hₘ_{₁}, Hₘ_{₂}]≠0 in nature. The problem is to determine the control fields such that any desired evolution can be achieved. The set of reachable states of the Hamiltonian H_{QOC} is connected with the dynamical lie algebra [symbol] of generators G which are defined by nested commutators of the set g that is spanned by Hamiltonians Hₘ,

$$G = [ {g}_{1} , [ {g}_{2} , … ] ] \qquad ( 10 )$$

where gᵢ∈g=span(Hₘ) and the lie group is the exponential map of this dynamical lie algebra,

$$𝒢 = {e}^{ℒ} = \{ {e}^{{A}_{1}} {e}^{{A}_{2}} {e}^{{A}_{3}} …… \text{:} {A}_{1} , {A}_{2} , {A}_{3} … . ∈ ℒ \} \qquad ( 11 )$$

This means that once the dynamical lie group is known, the reachable states of the system are known. A connection can be established between the reachability of an ansatz in a VQA and the dynamical lie algebra. They use the notion of the lie rank criterion which is the dim( [symbol] ) to study the reachability and claim that higher lie rank will result into a better ansatz for optimization. This ansatz is build by selecting operators from the lie group shown in Eq. (11).

There is a close connection between how the terms in the dynamical lie group [symbol] and the CD pool operators A_{λ} are calculated. Both of them are calculated by executing nested commutator operations of non-commuting control Hamiltonians. Hence, similar to the criterion of QOC, the circuit ansatz consisting of terms only from the nested-commutators will be a good ansatz. Adding more non-commuting terms will improve the results significantly and is done in some embodiments of the invention.

Apart from that, an important task is to choose the parameterization of the ansatz. In this example, the method is restricted with only a few terms of the NC method, having each terms their own free parameters will be a clever idea as this will also help in increasing the degrees of freedom of the ansatz. At this point, it is worthwhile to point out that there has been a recent study that utilizes spatial symmetries to determine the parameterization where they find the automorphism group of the graph so that the graph symmetries will help in finding suitable parameterization for the ansatz.

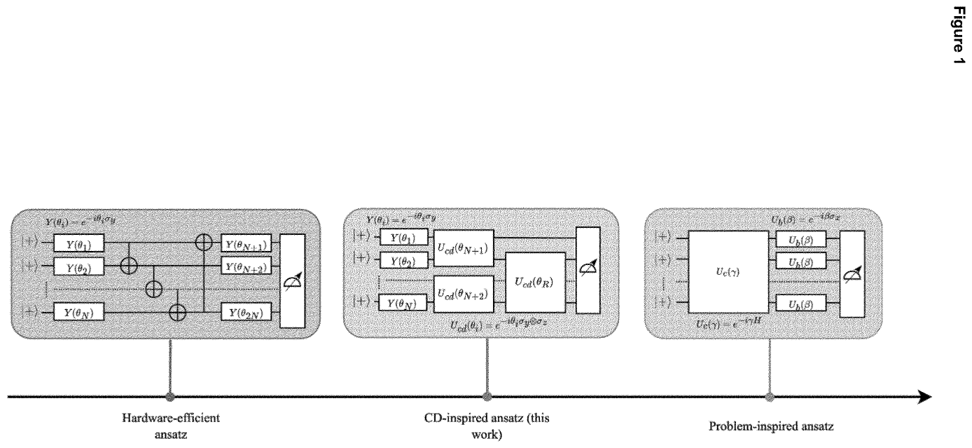

Summing up, the circuit ansatz will be initialized in the |+ [symbol] ^{⊗N} state and then it will consist of trotterized terms from the pool of operators obtained from Eq. (8) with each term having its free parameter to be optimized by classical optimizer. The number of free parameters is dependent on the number of interaction terms in the Hamiltonian. A schematic diagram of hardware-efficient ansatz, CD-inspired ansatz and problem-inspired ansatz is shown in FIG. 1. In the next section, w the protein folding problem is reviewed and the ansatz depending upon the problem is constructed in an exemplary fashion.

### V. Protein Folding

In the quantum computing regime, protein folding is tackled as a lattice problem where amino acids (of which proteins are built) are sequentially added to a given lattice such that the total conformation energy is minimum. The lattice can be 2D (plane) or 3D (for example, cubic or tetrahedral). The complexity increases exponentially with increasing dimensions because of the highly increasing number of possible configurations a protein can have. After selecting the lattice structure, the next important step is to choose the type of encoding. The widely studied encoding types are position encoding, where the lattice coordinates are encoded as qubits, and turn encoding, where the turn direction is encoded as qubits. In position encoding, the solution bitstring will represent the coordinates where the respective amino acids should be placed for energy minimization, and, for turn encoding, the solution contains information about the turns taken by each amino acid sequentially to result in minimum energy configuration. The difficulty and the form of the Hamiltonian are highly dependent on the type of encoding chosen. Another vital task is to assign each amino acid's interaction energies that should be minimized at the end of the algorithm. Mainly there are two types of interactions namely, the Hydrophobic model (HP), where the interaction coefficients are limited to only two values and the Miyazawa and Jemigan (MJ) interaction where the coefficients are arbitrary depending upon the contact of amino acids. Lastly, depending upon the structure, one may define some constraints that will help avoid configurations that are not allowed, for instance, stacking different amino acids on the same lattice point.

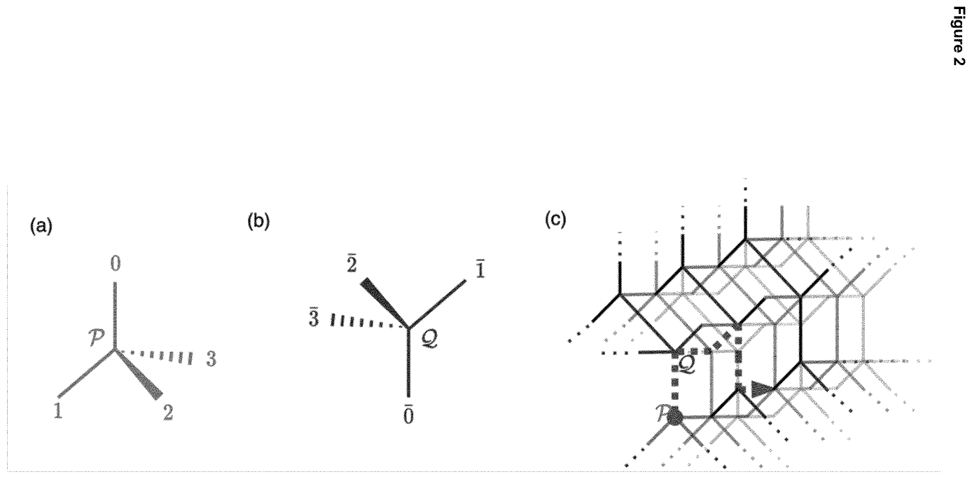

In this embodiment, the 3D tetrahedral lattice model is chosen with the turn encoding and MJ interactions. The qubits are turn encoded so that each of the encodings represents the turn tᵢ taken by the (i+1)^{th} amino acid after i^{th} amino acid. There are two sets of lattice P and Q each exactly opposite to each other where the possible four turns are shown by t=0, 1, 2, 3 (0̅, 1̅, 2̅, 3̅) and each of the numbers shows the different direction of the tetrahedron. Here, two qubits are devoted to encode each turn so that the turns are given by tⱼ=qⱼ/qⱼ₊₁ which scales as 2(Nₐ−3) where Nₐ is the number of amino acids considered. P and Q are changed at every turn so that even turns can be shown by P and odd turns can be shown by Q. An example of the turn, bitstring will be of the form t=01̅13̅20̅ . . . etc. The schematic diagram is shown in FIG. 2. The initial two turns can be fixed to t₁=01 and t₂=00 due to the symmetry of space considered here. In addition, one more qubit q₆=1 can also be saved due to symmetry. Hence, the conformation qubits Q_{c} will look like

$${Q}_{c} = [ 00 ] [ 01 ] [ {q}_{5} 1 ] [ {q}_{7} {q}_{8} ] ... .. [ {q}_{2 ( N - 1 ) - 1} {q}_{2 ( N - 1 )} ] \qquad ( 12 )$$

To keep track of the turns, a function gₘ where m=0, 1, 2, 3 is constructed and this function will return 1 if the axis a is selected at i^{th} turn. The shortest distance between any two beads can be found by keeping track of the number of turns the beads had taken with lattices P and Q. As far as the constraints are concerned, there are two constraints namely, growth constraints that penalize unwanted conformations and chirality constrain that enforce correct chirality. To impose these, two terms H_{gc}(Q_{c}) and H_{ch}(Q_{c}) are added to the problem Hamiltonian with positive Lagrange multipliers (θ_{gc}, θ_{ch}). Details about how to create these functions are known in the art. Along with Q_{c}, a set of qubits Qᵢₙ are included that takes account of the interactions between the nearest neighbor beads in the protein chain. Qᵢₙ=qᵢⱼ are a set of two-indices qubits that have information about nearest neighbor contact with i^{th} and j^{th} bead. If the contact occurs the energy eᵢⱼ is applied to the Hamiltonian. Thus, the total qubits Qₜₒₜ={Q_{c}, Qᵢₙₜ} and the Hamiltonian Hᵢₙ is given by,

$$H ( {Q}_{tot} ) = {H}_{gc} ( {Q}_{c} ) + {H}_{ch} ( {Q}_{c} ) + {H}_{in} ( {Q}_{int} ) \qquad ( 13 )$$

H(Qₜₒₜ) is a 5-local Hamiltonian whose ground state will be the solution to the protein folding problem. The specific reason for the selection of this model for benchmarking the CD-inspired ansatz is that this model has a tetrahedral lattice that captures many physical and chemical properties and this model uses a lower number of qubits for a specified amino-acid chain, to solve the problem as compared to other methods but this comes at the cost of increasing the locality of the Hamiltonian. In the next sections, it is shown that this increased locality does not affect our CD-inspired ansatz and the optimal solutions can be obtained by using only 2-local terms in the PQC which is in contrast to QAOA where the circuit ansatz will require 5-local terms in the ansatz which makes it practically impossible to implement on a real device.

### V. Numerical Simulations

In this section, the CD-inspired ansatz is applied to various proteins with different numbers of amino acids. These include the amyloid-beta peptide sequence (KLVFFA) which translates to a 6-qubit system, the Neuropeptide-alpha bag cell (APRLRFY), which translates to a 9-qubit system, cyclic peptide inhibitor (AVDINNNA) which translates to a 13-qubit system and Oxytocin (CYIQNCPLG) which translates to a 17-qubit system. The interactions between these amino acids are the MJ interactions.

In each case, a 5-local Hamiltonian is generated intending to reach the optimal bit-string (ground-state of the Hamiltonian) that shows the protein configuration by minimizing the expectation value. By implementing the NC commutator as given in Eq. (5), a set of CD terms is obtained with increasing locality, out of them, second order CD terms are chosen heuristically; Y+YZ where

$$Y = {e}^{- i {\sum }_{m} {σ}_{m}^{y}}$$

where m=0, . . . , N, and

$$YZ = {e}^{- i {\sum }_{i , j} {J}_{ij} {σ}_{i}^{y} {σ}_{j}^{z}}$$

where (i, j) correspond 2-body interactions that are present in the problem Hamiltonian and Jᵢⱼ are the coefficients of those 2-body interactions. The number of 2-body terms N_{2b}≤N(N−1)/2 make this ansatz hardware-efficient in the sense that as compared to the problem-inspired ansatz like QAOA, its experimental implementation is much more feasible. Regarding the optimizable parameters, each of the gates that are applied has its free parameter. Hence, the number of parameters per layer is R=N_{2b}+N where N is the system size so the parameter scaling is [symbol] (N²). The parameterization can be shown as a function of the system size of HEA which is R=2N, CD-inspired ansatz for protein folding, and CD-inspired ansatz for a extreme case where all-to-all interactions are present which is R=N(N−1)/2+N.

For the classical optimization part, stochastic gradient-descent-based optimizers called Adam optimizer and Adagrad optimizer are implemented in this example. For each protein, the algorithm was run 20 times with different random initial parameters for the p=1 layer. To quantify the performance of the algorithm, “success probability (S)” was used as a metric that shows the probability of getting the ground state at the end of the algorithm, and the expectation values were studied as a function of the iterations steps to understand the convergence. To minimize the number of measurements during the optimization process, as the Hamiltonian is classical, one can use grouping of commuting Pauli terms to find the expectation values using less measurements.

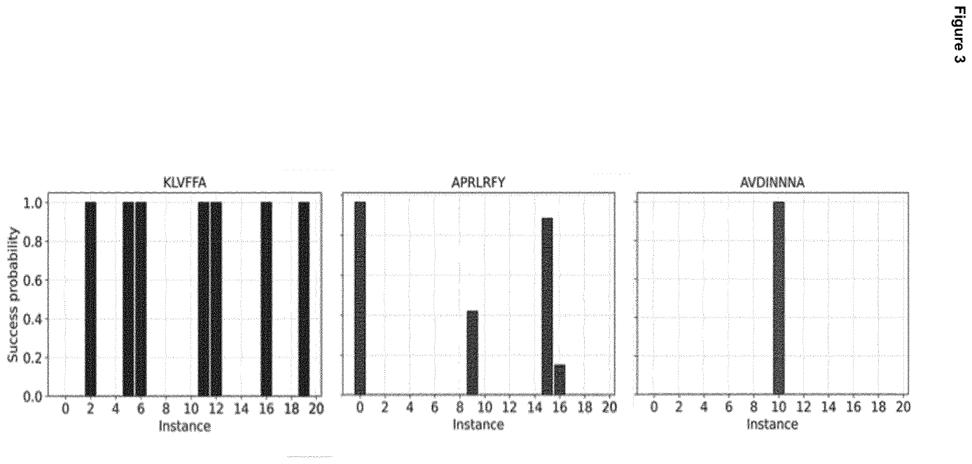

FIG. 3 shows the success probabilities for p=1 CD-inspired ansatz as a function of the number of instances where for each instance, the ansatz was randomly initialized. One can see that the ansatz performs extremely well for all the cases investigated. With increasing system size the number of successful instances decreases, this is due to the fact that with increasing system size, the number of terms in the Hamiltonian increases drastically and so it becomes difficult to explore the Hilbert space which increases the importance of choosing suitable initial parameters. Nevertheless, the overall performance of this ansatz is commendable because only local optimizers were used, and the ansatz had only p=1 layer. For the numerical simulations, the Adam optimizer was used with step size=0.1 for 6-amino acid, the Adagrad optimizer with step size=0.05 for 7-amino acid protein, and the Adam optimizer with step size=0.1 for 8 amino acid protein. The number of iterations was set to 500 with the tolerance of 10⁻⁶ in the sense that if the consecutive expectation energy values are less than the tolerance value, the iterations stop to give the final result. In all the instances where the initialization is optimal, the algorithm ends up in the global minimum, giving very high success probabilities. Also, the ‘unsuccessful’ instances show almost zero success probability, this behavior can be attributed to the fact that in the spectrum of energy eigenstates, the excited states are very close to the ground state so there is a finite possibility of the output state to be stuck at the first or second excited state with high probability.

Concerning another problem-inspired ansatz like QAOA, CD-inspired ansatz is better in two ways. First, the problem Hamiltonian for this problem is 5-local, which makes the Hamiltonian term extremely difficult to implement in terms of the gate-based model. Another important fact is that QAOA has only two parameters per layer which make it very less expressible and thus a high-layered ansatz will be required to contain the solution.

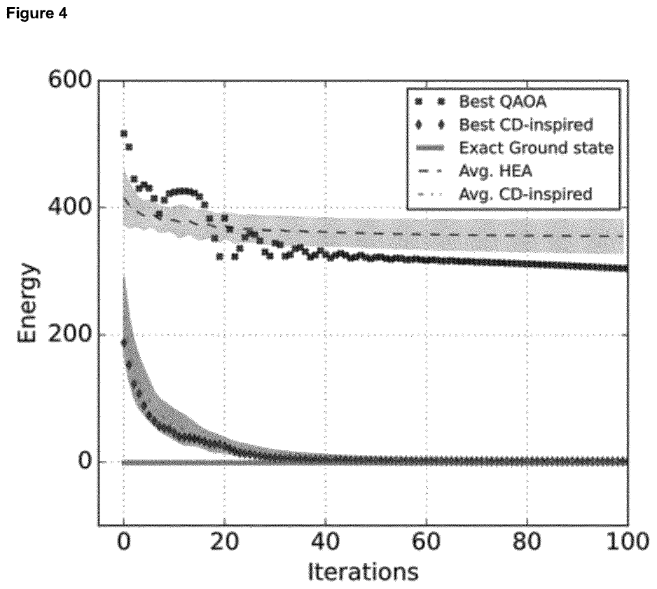

To compare QAOA with CD-inspired ansatz, the convergence of the best out of the 20 instances was studied for both CD-inspired ansatz and QAOA, the results are shown in FIG. 4. Energy as a function of iteration steps is plotted and compared with the exact ground state energy. One can see that when the initialization is near the global minimum, the classical optimizer works smoothly to take the energy to the global minimum. One can also see that in a very low number of iteration steps, one can successfully get very close to the ground state which turns out to be an important factor when experimental implementation is considered.

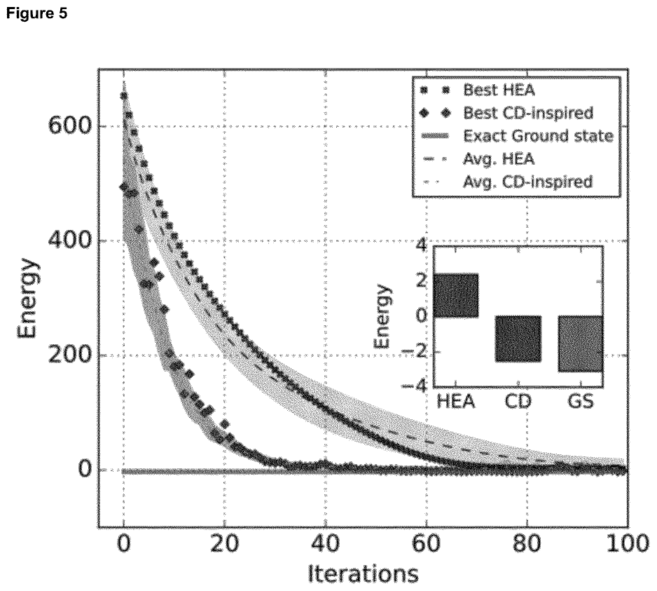

To compare the performance with the hardware-efficient ansatz (HEA), the inventors implemented a circuit with parameterized Y rotation applied to all qubits followed by CNOTs to the cyclic nearest neighbors for entanglement, followed by parameterized Y rotation applied to all qubits. In FIG. 5, a nine amino-acid protein was studied that translates to a 17-qubits system. Results show a comparison between the energies as a function of iterations for 100 iterations and using the Adam optimizer with step size=0.03. These results are also the best out of 20 instances with randomly chosen initial parameters. The inset plot shows the minimum energy obtained during the optimization. The minimum when HEA is applied is much higher than CD-inspired ansatz, and its minimum is much closer to the exact ground-state energy with CD-inspired ansatz. Regarding the convergence with iteration steps, the HEA has a relatively smoother convergence as compared to the CD-inspired ansatz and also with 100 iterations and the step size considered, the algorithm with CD-inspired ansatz is not converging to a steady energy value. The reason behind that might be the relatively large step-size and a higher number of iteration steps may be needed to reach to convergence. This also can mean that near the ground-state, the energy landscape is extremely featured which makes it harder for the local optimizer to converge to a particular energy value. Also, the energy difference between the ground state energy E_{GS} and the minimum energy obtained from the CD-inspired ansatz E_{cd} is E_{cd}−E_{GS}≈0.54. Thus, even at a large system size, solutions are obtained very close to the ground-state which is also helpful as variational algorithms aim to find approximate solutions that are near to the ground state.

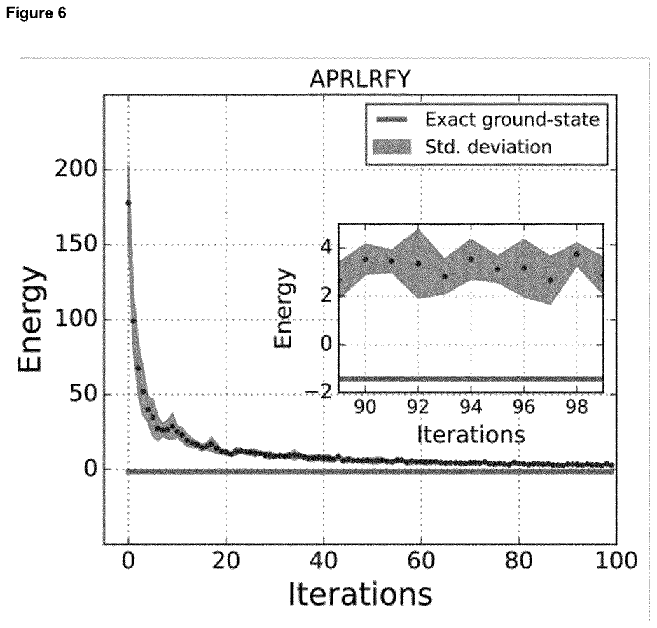

In order to implement the algorithm against noise, the inventors created a noise model which mimics the noise of ibmq_guadalupe device from IBM. This noise model uses the actual backend parameters to create a noise model with some assumptions. The cross-talk errors and leakage errors are not included in the model. Noisy simulations are key in understanding how the energy landscape changes under the effect of noise. In FIG. 6, the optimal initial parameters (of ideal simulation) of a N=9 qubit system with p=1 were taken and the algorithm performed against the noise model five times and plot the average energy as a function of iterations. The aim is to check how much noise changes the convergence of the optimal algorithm.

one can observe that the ansatz works considerably well against the noise model for 100 iteration steps. Another observation is that the final convergence energy is lifted considerably as compared to the ground state energy and the algorithm is not exactly converging; there are still fluctuations when reaching the end. This behavior is natural due to existing noise. This algorithm was performed with Adagrad optimizer as a classical optimization routine. This gives an intuition about the energy landscape when CD-inspired ansatz is implemented. From the convergence, it is evident that the energy landscape is overall smooth but there exist lots of local minima near the exact ground state. These landscape features make it easier for the classical optimizer to get near to the ground state but to reach exactly to the ground state becomes difficult with increasing system size. This fact is also evident from FIG. 3, as with increasing system size, the number of successful instances decreases. Hence, at higher system sizes, there might be a requirement for global optimizations to efficiently reach the ground state with a high success probability. Also, the noisy simulations were run without any error mitigation techniques. The involvement of error mitigation techniques are crucial at the time of experimental implementation. There has been significant attention in studying error mitigation techniques. Also, recently an algorithm was proposed to mitigate transient errors while navigating the energy landscape of VQAs.

With the advantages of implementing this ansatz, several challenges need to be addressed. First is the sensitivity to initial parameters. The performance of the ansatz depends upon the initial parameters chosen. Without the knowledge of the suitable initial parameters, the scaling would be difficult as at higher system sizes, the randomized initialization might require many instances to find appropriate parameters. Secondly, the choice from the pool of CD terms in this work is heuristic but clever techniques need to be developed for choosing the CD term that perform the best depending on the problem. Finally, from the results, it can be seen that when the ansatz does not perform well, the results are close to zero, which means that when scaling to higher system sizes, it might be difficult to find the exact ground state as the size of the Hilbert space will be huge. To circumvent these challenges, machine learning can be used.

### VI. Experimental Implementations

In this section, the proposed CD-inspired ansatz is implemented on different available noisy hardware and emulators, specifically on trapped-ions and superconducting systems. Having various native gate sets and connectivity, all these devices pose different challenges that require to be dealt with to get appreciable results. Details about the hardware, circuit optimization, and error mitigation techniques are given in the section “Error mitigation techniques for IBM implementation”.

### A. Quantinuum Trapped-Ions

Two systems were used as examples, 8 amino acid protein AVDINNNA (N=13 qubits) and 9 amino acid protein CYIQNCPLG (N=17 qubits) with p=1 layers on a Quantinuum H1-1 device. For the trapped-ions system, all the qubits are identical and errors depend upon the interaction zones, so the selection of qubits becomes trivial since one can choose any. In both cases, the system was initialized in the |+ [symbol] ^{⊗N} state by applying Hadamard gates to all the qubits. Following that, parameterized R_{y}(θᵢ) rotations were implemented on all qubits, and the interaction terms YZ(θ) were constructed by the native ZZ(θ) interaction. This is done by applying two rotations,

$${YZ}^{ij} ( {θ}_{m} ) ≡ {R}_{x}^{i} ( π / 2 ) {ZZ}^{ij} ( {J}_{ij} {θ}_{m} ) {R}_{x}^{i} ( - π / 2 ) . \qquad ( See FIG . 10 )$$

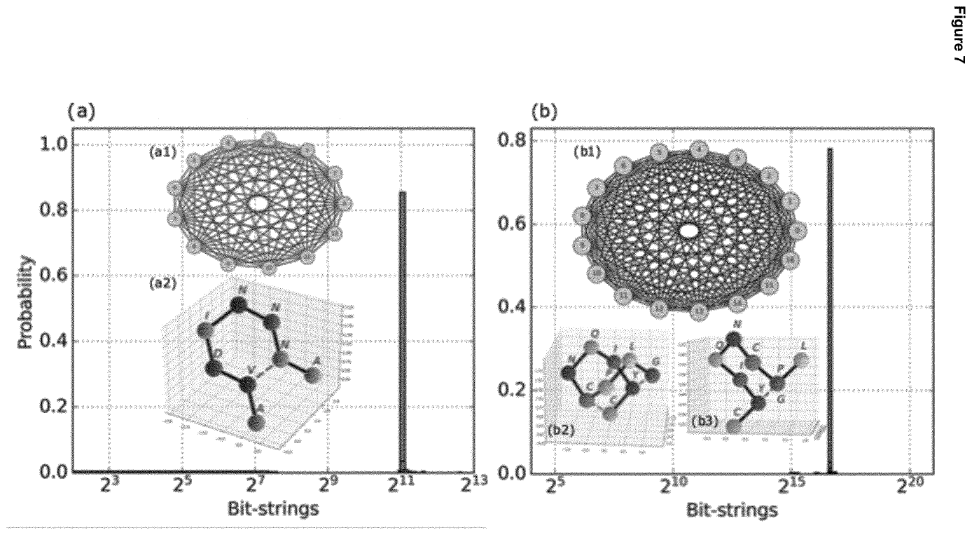

For N=13 system, the Hamiltonian considered had N_{2loc}=52 two-body interactions and for system with N=17 qubits, N_{2loc}=80 two-body interactions were present. Graphs in FIG. 7(a1) and FIG. 7(b1) show the full connectivity with blue edges showing the present interactions and purple edges showing the interactions that are absent. When dealing with the real hardware, gates are applied in a parallel manner to reduce the circuit depth but this comes at a cost of performing multiple operations at the same time. In this system, there is a limit on how many parallel operations can be performed efficiently, so the gates are applied such that they do not exceed 5 operations at a time. Due to the limited hardware access, the optimization part was run on a local simulator with Adam optimizer and 500 iteration steps and implemented the circuit obtained with the optimal parameters on the real hardware.

In FIG. 7, the probability distribution is shown from the real hardware for both systems. Both experiments were performed with 1000 shots. For N=13 qubits, around 85% probability was achieved of getting the ground state shown in FIG. 7(a), while FIG. 7(a2) shows the folded protein in 3D. The nearest-neighbor amino acid connection is ‘V’-‘N’, which gives us q_{2,7}=1. The optimal turn sequence is tₑₓₚₜ=[1̅, 0, 1̅, 2, 0̅, 1, 0̅]. On the other hand, FIG. 7(b) shows the N=17 qubits case with around 79% probability of the output state achieved during the experiment which is the 4th excited state of the Hamiltonian. As the system size is large and only p=1 layered ansatz is implemented, the classical optimization routine gets close to the ground state energy but still reaches local minima. FIG. 7(b2) shows the protein configuration with the ground state, while FIG. 7(b3) is the protein configuration with the fourth excited state. The optimal turn sequence is tₒₚₜ=[1̅, 0, 3̅, 1, 0̅, 2, 1̅, 3] and the turn sequence obtained while experimenting is tₑₓₚₜ=[1̅, 0, 3̅, 2, 0̅, 3, 1̅, 1]. The last turn taken by the amino acid is non-optimal which leads to overlap at the second last lattice point. For optimal configuration, the nearest-neighbor connections are ‘C’-‘C’ and ‘Y’-‘G’, and hence q_{1,6}=1 and q_{2,9}=1. However, with the 4th excited state the connections are ‘Y’-‘G’ and ‘Y’-‘P’, hence q_{2,9}=1 and q_{2,7}=1. So, for the fourth excited state, there is one connection and several turns that differ from the exact ground state. In terms of the energy difference, both these configurations differ by a factor of 0.54 (arbitrary units). This is close to the actual ground state and, thus, the algorithm is likely to jump to the excited state. Implementation of our hybrid quantum algorithm results in high success probability. This is due to the all-to-all connectivity and the fact that the two-body interactions in the circuit can be applied effectively with native gates.

### B. QVM

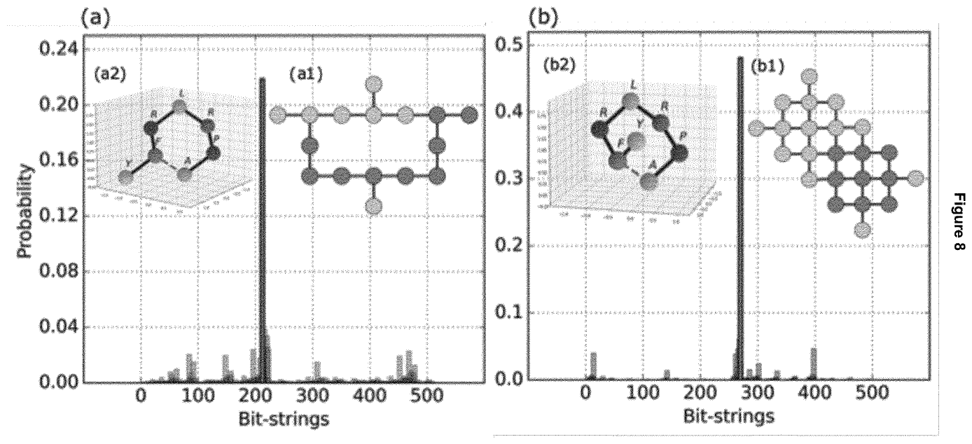

In FIG. 8b, the output probability distribution is shown with Google's QVM rainbow of a N=9 qubits optimal circuit and 10000 shots. The real hardware from Google is called ‘Sycamore’ which has 53 usable transmon qubits arranged in a 2D grid. Hence, each physical qubit is connected to at most 4 other qubits. The single-qubit gates are executed by microwave pulses of a fixed frequency and two-qubit gates can be executed by bringing the nearest qubits on resonance and then turning on a coupling. The two-qubit native gates are the controlled-Z, \sqrt{iSWAP}, and Sycamore interactions. As per the datasheet published on May 14, 2021, the typical two-qubit \sqrt{iSWAP} implementation error is 1.4% per gate when applied in parallel and the typical single-qubit gate error is 0.1% per gate.

The inventors performed the experiment in the QVM offered by Google. Google offers two QVMs: rainbow and weber. In QVM, a noise model is implemented that closely mimics the actual noise of the hardware. QVM rainbow is a 23-qubit device with square-grid lattice connectivity. The inventors performed the experiment with N=9 qubits system APRLRFY. As usual, start with the |+ [symbol] ^{⊗N} state by applying Hadamard gate to all the qubits. Then, R_{y}(θᵢ) is applied where i=0, 1, 2, . . . 8. For the experiment, the inventors have selected a square grid of 3×3 qubits to perform the algorithm and CZ as a native two-qubit gate. To implement the YZ interaction, one needs to create a ZZ interaction first using CZ gate which is accomplished by

$${ZZ}^{ij} ( {θ}_{m} ) ≡ {H}^{j} {CZ}^{ij} {H}^{j} {R}_{z}^{j} ( {J}_{ij} {θ}_{m} ) {H}^{j} {CZ}^{ij} {H}^{j}$$

where m=9, 10, . . . , R. In this case N_{2b}=25 that means R=34. From here, YZ^{ij}(θₘ)≡Rₓ(π/2)ZZ^{ij}(θₘ)Rₓ(−π/2). As mentioned before, the interactions are the ones present in the Hamiltonian. As the connectivity is not all-to-all, a SWAP strategy is required to implement the circuit with greater efficiency. For this circuit, a SWAP strategy was applied that implements the whole circuit by using 11 SWAP gates. As all the 2-qubit interactions are not present in the circuit, one can remove some redundant SWAP gates to further optimize the performance. The optimization was run with this circuit on a local simulator and obtained the optimal parameters Θᵢ as before.

To have the best efficiency, it is also necessary to optimize the ‘moments’ of the quantum circuit. A ‘moment’ is defined as a set of operations, acting on different qubits such that all these operations can be applied at a single abstract time slice. After obtaining the θᵢ, the inventors also optimized the circuit such that the number of ‘moments’ are reduced. In other words, the circuit was arranged in such a way that the number of operations that can be applied at a single time is maximized. There are several pre-defined ways to do this, for example, aligning the circuit to the left, i.e. maximum possible number of operations are arranged from the start of the circuit. For the exemplary implementation, the circuit where all the operations are aligned into similar categories was optimized. The circuit was selected in such a way that all the single-qubits and two-qubit operations are aligned in separate ‘moments’. After including all these optimizations, a success probability of around 48% is achieved. Even for a N=9 qubits system, the results are lower as compared to the trapped-ions system. This behavior is mainly due to the circuit decomposition and the connectivity of the system. This probability is reduced when applying it to their real device. Improving these results can be achieved by testing various combinations of native gates to generate YZ interactions, also implementing more techniques like error mitigation and dynamical decoupling.

### C. Superconducting Chip

Finally, the inventors implement the algorithm in the IBM superconducting chip. IBM systems are fixed-frequency transmon qubits and the gate operations are done using microwave pulses. Implementation in IBM devices require several additional strategies as the qubit connectivity selected is linear. The N=9 qubit problem was implemented on ibmq_guadalupe device which is a 16 qubit device. Starting with applying a Hadamard gate to all the qubits followed by parameterized R_{y}(θᵢ) rotations where i=0, 1, . . . , N. The two-qubit native gate for this device is the cross-resonance gate R_{zx}(θ) also known as CR. The two-qubit gate decomposition is given by

$${YZ}^{ij} ( {θ}_{m} ) ≡ {R}_{z}^{i} ( - π / 2 ) {CR}^{ji} ( {J}_{ij} {θ}_{m} ) {R}_{z}^{i} ( π / 2 )$$

where m=9, 10, . . . , R and i shows the control qubit and j shows the target qubit and R=34. These gates were arranged in parallel before the optimization process such that the resultant circuit is as shallow as possible. This circuit was optimized and find the optimal parameters Θᵢ found and this circuit implemented on the hardware. This implementation requires additional strategies for choosing the best layout in certain embodiments, such as SWAP strategies for achieving the desired connectivity, native gate and pulse optimization strategies, dynamical decoupling, and finally measurement error mitigation techniques. Details about these techniques are given in the section “Error mitigation techniques for IBM implementation”.

As seen from FIG. 8a, the inventors achieved a ground state probability of around 20% even though after the improvements done for this hardware, the effect of noise reduces significantly. These results are considerably good as the connectivity used in this embodiments is only linear.

### VII. Discussion

In this example, the inventors implemented a hybrid digitized counterdiabatic quantum algorithm to investigate protein folding problem. The parameterized quantum circuit associated with this algorithm is inspired by counterdiabatic driving, has [symbol] (N²) parameterization and consists of only one qubit and two-qubit gates. The inventors applied this algorithm to the protein folding problem that is encoded in a Hamiltonian such that the ground state contains the information about the configuration of the required protein. Due to the encoding the inventors chose, this Hamiltonian is a 5-local classical Hamiltonian. the inventors study various proteins with increasing amino acid chains and show that the ansatz performs exceptionally well and it is also experimentally feasible to implement it on real hardware with various strategies. The ansatz was implemented to several quantum hardware like trapped ions and superconducting chips.

### Error Mitigation Techniques for IBM Implementation

### 1. Best Layout

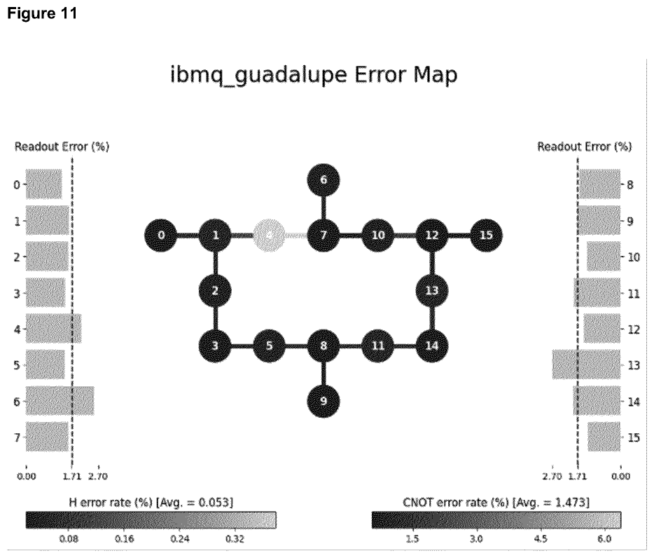

Qubit selection is a crucial task while implementing the circuit as each qubit possess their own single-qubit gate errors and two-qubit gate errors with the connecting qubits. To select the best qubits from the device, a sub-graph isomorphism algorithm was implemented which selects 9 qubits out of 16 that minimize the expected gate errors (see FIG. 11 for the IBM hardware layout chosen for the experiment).

### 2. Swap Strategies

IBM's heavy-hex connectivity scheme means there only exists nearest neighbour coupling. With a linear connection of qubits, one need a SWAP strategy to implement the circuit using as low number of SWAP gates as possible. As some of the two-qubit interactions are missing, the connectivity could be covered by applying 30 SWAP gates. For layered schemes like in the case of QAOA, this layout fitting introduces additional instructions and, therefore, increases the noise affecting the final result by the error rate addition for those additional instructions. Shallower ansatz may become handy given this fact, as the shallower the circuit is less SWAP gates will be introduced.

### 3. Native Gate Compilation

SWAP, CNOT and ZZ gates are not native to IBM's chips, being translated to the native CR gate before this circuits are executed. These native gates can be translated beforehand so by the annihilation of commuting gates the depth of the final logical circuit can be reduced even further. By being hardware-specific in this regime, one can also calibrate those very same CR gates so that no further translation is done and, therefore, the actual physical coupling represents intended interaction gate between two qubits. Hence, the final pulse schedule gets reduced to its minimal expression according to the specifications of the hardware while preserving the intended structure for the ansatz. Once the circuit is reduced to its minimum expression, there are still some techniques one can apply for noise mitigation in certain embodiments, some encoded within our pulse definition and other by statistically correcting the systematic error upon measurement.

### 4. Dynamical Decoupling

Dynamical decoupling techniques introduce different gate schemes to minimize the noise generated by the idling effect of the qubits while other longer-time two-qubit gates are being applied. They observed that even though unnecessary, if idling time is used applying gates that will not change the final result of the computation being done, the effect of noise gets reduced significantly. Pretty known is the XY4 scheme that basically applies a sequence of four rotation operation on X and Y axis. Following a symmetrized version of the XY4 scheme, the XY8 scheme was introduced into our experiment runs shown by

$${X}_{π} \to {Y}_{π} \to … \to {X}_{- π} \to {Y}_{- π}$$

where −π and π reflect the opposing pulse amplitude being applied on each axis. Thus, the result of the gate application renders the I only that by the active involvement low-frequency noise generated while idling gets reduced.

### 5. Measurement Error Mitigation

Finally, readout error can be seen as systematically and consistent for a given chip, always representing a similar error distribution along the usage of a chip. By compensating this final measurement error one could boost the final results to their statistically corrected error-less version. A matrix-free approach can be used where for a given ideally simulated probability distribution \vec{p}_{ideal}, upon measurement on a real device would yield a noisy distribution \vec{p}_{noisy} such that

$${\vec{p}}_{noisy} = A {\vec{p}}_{ideal} ,$$

where A represents a matrix translating this change on the bit-string outcome probability between one another. By solving this equality and finding out the matrix one could then recover the ideal case from a noisy distribution, enhancing the obtained probabilities.

Although illustrative embodiments of the present invention have been described herein with reference to the accompanying drawings of the figures, it is to be understood that the invention is not limited to those precise embodiments, and that various other changes and modifications may be made by one skilled in the art without departing from the scope or spirit of the invention.

1. A computer-implemented method for solving an optimization problem for the folding of a protein with a given amino acid sequence, wherein a problem Hamiltonian encoding the problem is to be minimized, comprising:
choosing a lattice model,
placing the amino acids of the protein sequence in the lattice model, minimizing an interaction energy of the amino acids, thereby creating a problem Hamiltonian, and
performing an optimization on the problem Hamiltonian to obtain a final Hamiltonian in a ground state reflecting the solution to the optimization problem.

2. The method of claim 1, comprising
calculating a set of couterdiabatic (CD) terms for an adiabatic Hamiltonian which comprises the problem Hamiltonian using a nested commutator method, and
selecting a set of operators from the set counterdiabatic terms for implementation on a quantum processor.

3. The method of claim 2, wherein the set of couterdiabatic (CD) terms comprises or consists of 2-local terms.

4. The method of claim 1, wherein the couterdiabatic terms are used to construct a variational quantum circuit in the form of a parametrized quantum circuit comprising a set of quantum gates.

5. The method of claim 4, wherein the parametrised quantum circuit comprises at least one variational parameter that is optimized using an optimizer, in an iterative fashion.

6. The method of claim 5, wherein at each iteration of the variational optimization, an energy expectation value corresponding to the problem Hamiltonian with respect to a trial state is measured and the at least one variational parameter is updated to minimize the expectation value.

7. The method of claim 6, wherein the energy expectation value of the problem Hamiltonian is defined as
$$〈 {H}_{p} 〉 = ( {ψ}_{trail} \vert {H}_{p} \vert {ψ}_{trail} 〉 ,$$
wherein Hₚ is the problem Hamiltonian and |ψₜᵣᵢₐₗ [symbol] is a trial wavefunction.

8. The method of claim 1, comprising measuring qubits in a computation Z basis after the variational optimization to obtain the expectation value over the ground state of the final Hamiltonian.

9. The method of claim 1, wherein
the protein folding problem is encoded into a computable higher order unconstrained binary optimization problem, and the lattice model is adopted to simulate the composition of the protein in a three-dimensional space, wherein the method comprises
coding binary amino acid sequences, defining a step length and coordinates of any amino acid in the coded binary amino acid sequences in a predetermined direction and a distance between any two amino acids,
constructing a corresponding Ising Hamiltonian by adding constraint conditions, and
evolving the Ising Hamiltonian by utilizing quantum annealing to obtain a final evolution result, wherein the energy optimal solution obtained through the evolution is the most stable conformation of the protein and the result to the problem.

10. Use of the method of claim 1 in quantum chemistry, for solving an optimization problem for the folding of a protein with a given amino acid sequence, wherein a Hamiltonian encoding the problem is minimized.

11. A system for performing the method of claim 1, comprising
a quantum processor,
a memory to save the results of measurements of expectation values of a final Hamiltonian, and
a classical processor for performing a classical optimization.

12. A data processing device comprising means for carrying out the method of claim 1.

13. A computer program product comprising instructions which, when the computer program product is executed by a computer, cause the computer to carry out the method of claim 1.

14. A computation device comprising: an interface for communicating with a quantum-processing unit; and one or more processors configured to perform the method of claim 1 using the quantum-processing unit.

15. A non-transitory computer-readable storage medium comprising instructions which, when executed by a computer, cause the computer to carry out the method of claim 1.
